(19) United States
(12) Patent Application Publication
(10) Pub. No.: US 2012/0316430 A1
(43) Pub. Date: Dec. 13, 2012

(54) RADIOLUCENT SURGICAL INSTRUMENTS

(75) Inventors: Thomas David Aldag, Wichita, KS (US); Andrea Christine Meyer, Wichita, KS (US)

(73) Assignee: CIBOR, Inc., Wichita, KS (US)

(21) Appl. No.: 13/157,785

(22) Filed: Jun. 10, 2011

(51) Int. Cl. A61B 5/05

(52) U.S. Cl. 600/424

(57) ABSTRACT

A radiolucent surgical instrument comprising an internal stiffener composed of a first composite material having a first modulus and an overmold component formed around at least a portion of the internal stiffener and composed of a second composite material having a second modulus. The first modulus may be greater than the second modulus. The overmold component may be formed around the internal stiffener via injection molding. The internal stiffener may be a structural laminate fabricated by composite ply lay-up and cure and may have a bending stiffness between approximately 10 MSI and 16 MSI. The overmold component may be a short fiber reinforced thermoplastic form and may have a non-uniform distribution of thickness. The surgical instrument may include, for example, retractors, forceps, elevators, dissectors, suction tubes, drill guides, or separators.

### BACKGROUND

Embodiments of the present invention relate to surgical instruments and a method of manufacturing surgical instruments.

Surgical instruments such as retractors used for prying tissue and organs aside during surgery must be strong and durable. Therefore, most surgical instruments are made of stainless steel or other similar metals.

Sometimes x-rays must be obtained of a patient during surgery. However, because X-rays can not penetrate through most metals, the surgical instrument typically must be removed so as to not obstruct the surgeon's view of the patient's bone or implant replacement. For example, during orthopedic and spine surgeries, surgeons typically have to remove stainless steel instruments from the fluoroscopy field to get a good x-ray picture. Unfortunately, repeated removal of the surgical instrument increases the length of surgeries and can increase the risk of nerve and tissue damage.

Another problem with stainless steel surgical instruments is they are typically made from sheet material and are locally flat at points where they contact patient tissue. This is undesirable because tissue contact pressures are increased at the corners and edges of the flat instruments, resulting in higher risks of tissue injury. Furthermore, stainless steel surgical instruments are highly reflective, which can be distracting or obstructive to surgeons, particularly under bright surgical lighting.

Many implants, especially bearing surfaces of implants, are highly-polished materials. It is critical that the polished surfaces of these implants do not get scratched during installation, because scratched implants should not be used. The use of steel surgical instruments during surgery increases the risk of inadvertently contacting and scratching the polished surfaces of the implants.

Accordingly, there is a need for a surgical instrument that overcomes the limitations of the prior art.

### SUMMARY

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present invention will be apparent from the following detailed description of the preferred embodiments and the accompanying drawing figures.

Embodiments of the present invention solve the above-mentioned problems and provide a distinct advance in the art of surgical instruments. Various embodiments of the invention include a radiolucent surgical instrument comprising an internal stiffener and an overmold component. The internal stiffener is composed of a first composite material having a first modulus. The overmold component is formed around at least a portion of the internal stiffener and is composed of a second composite material having a second modulus. The first modulus may be greater than the second modulus. The overmold component may be formed around the internal stiffener via injection molding and/or may be joined with the internal stiffener via welding.

The internal stiffener may be a structural laminate fabricated by composite ply lay-up and cure and may have a bending stiffness between approximately 10 MSI and 16 MSI. In some embodiments of the invention, the overmold component may be a short fiber reinforced thermoplastic form. The overmold component may have a non-uniform distribution of thickness. The surgical instrument may be used as a retractor, such as a Hohman retractor, a Hibbs retractor, a Taylor retractor, or a Sweetheart retractor.

A method of fabricating a radiolucent surgical instrument may comprise the steps of forming an internal stiffener via composite ply lay-up and cure, placing the cured internal stiffener into a mold, and injecting composite material in liquid form into the mold and around the internal stiffener to form an overmold component of the surgical instrument. The method may then comprise the steps of hardening the injected composite material and removing the mold from around the overmold component.

### BRIEF DESCRIPTION OF THE DRAWING FIGURES

Embodiments of the present invention are described in detail below with reference to the attached drawing figures, wherein:

FIG. 1 is a perspective view of a Hibbs retractor type surgical instrument constructed in accordance with an embodiment of the invention;

FIG. 2 is a perspective view of an internal stiffener of the surgical instrument of FIG. 1;

FIG. 3 is a perspective view of an overmold component of the surgical instrument of FIG. 1;

FIG. 4 is a perspective view of a Taylor retractor type surgical instrument constructed in accordance with an embodiment of the invention;

FIG. 5 is a perspective view of an internal stiffener of the surgical instrument of FIG. 4;

FIG. 6 is a perspective view of an overmold component of the surgical instrument of FIG. 4;

FIG. 7 is a perspective view of a Hohmann retractor type surgical instrument constructed in accordance with an embodiment of the invention;

FIG. 8 is a perspective view of an internal stiffener of the surgical instrument of FIG. 7; and

FIG. 9 is a perspective view of an overmold component of the surgical instrument of FIG. 7.

The drawing figures do not limit the present invention to the specific embodiment disclosed and described herein. The drawings are not necessarily to scale, emphasis instead being placed upon clearly illustrating the principles of the invention.

### DETAILED DESCRIPTION

The following detailed description of the invention references the accompanying drawings that illustrate specific embodiments in which the invention can be practiced. The embodiments are intended to describe aspects of the invention in sufficient detail to enable those skilled in the art to practice the invention. Other embodiments can be utilized and changes can be made without departing from the scope of the present invention. The following detailed description is, therefore, not to be taken in a limiting sense. The scope of the present invention is defined only by the appended claims, along with the full scope of equivalents to which such claims are entitled.

In this description, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the present technology can include a variety of combinations and/or integrations of the embodiments described herein.

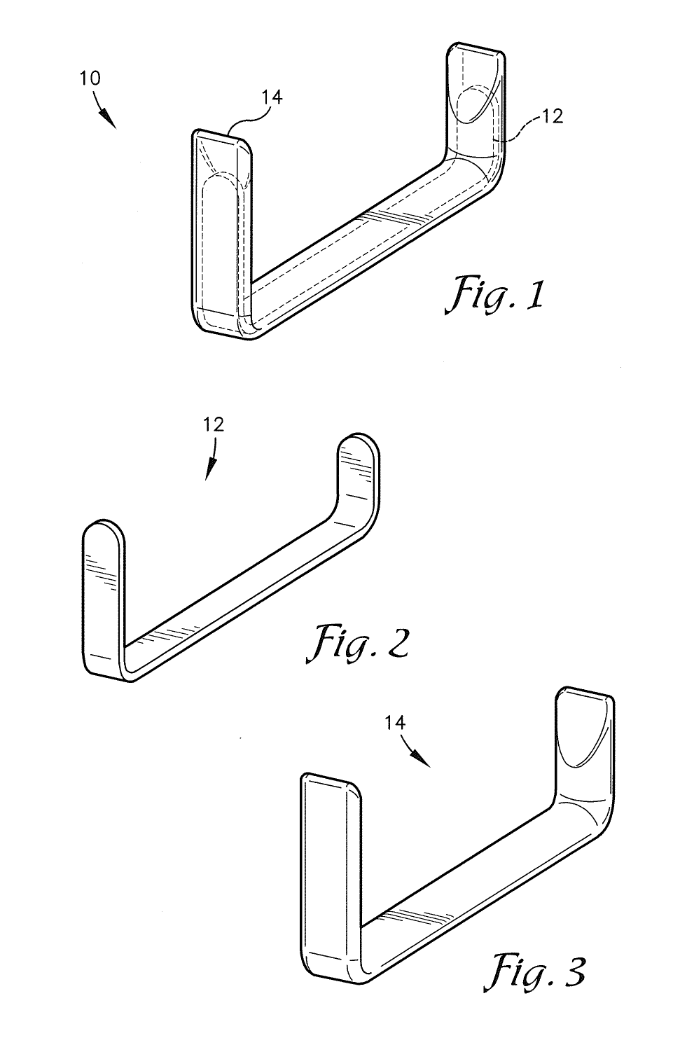

The present invention comprises different embodiments of radiolucent surgical instruments made of composite material. One such surgical instrument 10 is illustrated in FIGS. 1-3 and is designed for use as a Hibbs retractor, which may be used to hold back skin and tissue during surgery. The surgical instrument 10 may comprise an internal stiffener 12 and an overmold component 14 positioned around and/or attached to at least a portion of the internal stiffener 12. Both the internal stiffener 12 and the overmold component 14 may be made of composite materials. In some embodiments of the invention, the modulus or stiffness of the internal stiffener 12 may be greater than the modulus or stiffness of the overmold component 14.

The internal stiffener 12 may be a stiffened composite part, such as a laminated structural blade or spine. In some embodiments of the invention, the internal stiffener 12 may be approximately 0.1 to 0.22 inches thick. The internal stiffener 12 may also be formed with a substantially uniform thickness throughout a length of the part. However, the internal stiffener 12 may have any dimensions required for a given application. In some embodiments of the invention, the internal stiffener 12 may be manufactured to have a bending stiffness similar to aluminum and titanium. For example, the internal stiffener may have a bending stiffness of approximately 10 MSI to 16 MSI.

The internal stiffener 12, as illustrated in FIG. 2, may be formed of multiple plies of composite material. The plies may be comprised of polymeric matrix and reinforcement components. For example, the internal stiffener 12 may be an intermediate modulus commercial grade (or higher) carbon fiber in a thermoplastic matrix. However, other types of fiber and matrix may be used without departing from the scope of the invention. In some embodiments of the invention, the fiber volume of the plies for the internal stiffener 12 may range from approximately 50% to 65% of the total volume. Orientations of the plies may be tailored to provide strength and stiffness in primary load directions, depending on the load requirements of the surgical instrument 10. The plies may be unidirectional and/or woven material.

The plies may be consolidated using various lay-up techniques. For example, the plies may be stacked on a tool (not shown) with an appropriate lay-up sequence and then cured via pressure and heat, thus consolidating and hardening the composite plies into the internal stiffener 12 having a desired size and shape. The internal stiffener 12 may be cured via a press, autoclave, or any other means known in the art. In some embodiments of the invention, the plies may be stacked, cured, and then trimmed therefrom into a final desired shape of the internal stiffener 12. Specifically, once the internal stiffener 12 is cured, it may be trimmed by way of milling or other techniques into its final shape.

The overmold component 14, as illustrated in FIG. 3, may be made of a composite material of the same family as the composite material of the internal stiffener 12 to assure good bonding and compatibility when the two parts are joined. For example, the overmold component 14 may be a short fiber reinforced thermoplastic form made by way of injection molding. For example, the internal stiffener 12 may be positioned within a mold (not shown) and then the material for the overmold component 14 may be injected into the mold and hardened therein, thereby forming the overmold component 14 around the internal stiffener 12. Additionally or alternatively, the internal stiffener 12 may be attached to the overmold component 14 via welding or other methods of bonding composites together.

The internal stiffener 12 and/or the overmold component 14 may also comprise an x-ray marker (not shown) formed therein or thereon. The x-ray marker may be made of any non-radiolucent material, such that the instrument may be located via x-ray. Note that the composite material used for the remainder of the internal stiffener 12 and the overmold component 14 is radiolucent, such that it does not obstruct views of a patient's internal parts when x-rayed.

The overmold component 14 may be ergonomically contoured, curved, or rounded at various corner and edges thereof. These contours may particularly be formed at locations of the overmold component 14 intended to contact tissue and/or bones of the patient, thus minimizing tissue bruising and damage.

As illustrated in FIGS. 1-3, the surgical instrument 10 in the Hibbs retractor configuration may comprise a lateral portion with a handle portion at one end thereof and a blade or working end at another end thereof. In some embodiments of the invention, the working end and the handle portion are interchangeable—the two options allow the surgical instrument 10 to be used as a shallow or a deep retractor. The handle portion and/or the blade portion may extend at substantially right angles relative to the lateral portion, with the corner formed at this right angle being substantially rounded, as illustrated in FIG. 1. Furthermore, side edges of the surgical instrument 10 may be substantially rounded. In some embodiments of the invention, as illustrated in FIGS. 1 and 3, the overmold component 14 may comprise substantially tapered ends.

The method of forming the surgical instrument 10 with the composite overmold component 14, as described above, may advantageously allow for a non-uniform distribution of thickness and flexibility. Fabricating the overmold component 14 via injection molding may allow for easy and efficient forming of a complex shape. Specifically, injection molding the overmold component 14 can provide for complex contours, tapers, and rounded edges without the need for complex tooling and detailed milling of the composite material. Inclusion of the internal stiffener 12 in the overmold component 14 may provide greater load-bearing strength to the surgical instrument 10 than injection-molded composites alone may provide. The present invention combines the strength of composite parts formed via lay-up techniques with the ease of forming complex shapes via injection molding to form a strong, radiolucent surgical instrument.

A method of fabricating the surgical instrument 10 may comprise the steps of forming the internal stiffener 12 via composite ply lay-up and cure, as described above. Then the method may comprise placing the cured internal stiffener 12 into the mold corresponding to any desired surgical instrument final size and shape. Next, the method may comprise injecting composite material in liquid form into the mold and around the internal stiffener 12 to form the overmold component 14. The method may then comprise the steps of hardening the injected composite material and removing the mold from around the overmold component 14. As noted above, the modulus of the internal stiffener 12 may be greater than the modulus of the overmold component 14.

In some embodiments of the invention, the method may further or alternatively comprise bonding the internal stiffener 12 with the overmold component 14 via welding or any other bonding method. For example, in some embodiments of the invention where only a portion of the internal stiffener 12 is encapsulated by the overmold component 14, the overmold component 14 may be separately molded via injection molding and then welded together with the internal stiffener 12, such as by way of welding or other post-cure bonding methods. In some embodiments of the invention, multiple overmold components 14 and/or multiple internal stiffeners 12 may be used to form a single surgical instrument, particularly if movable joints or fittings are required for a particular surgical instrument design.

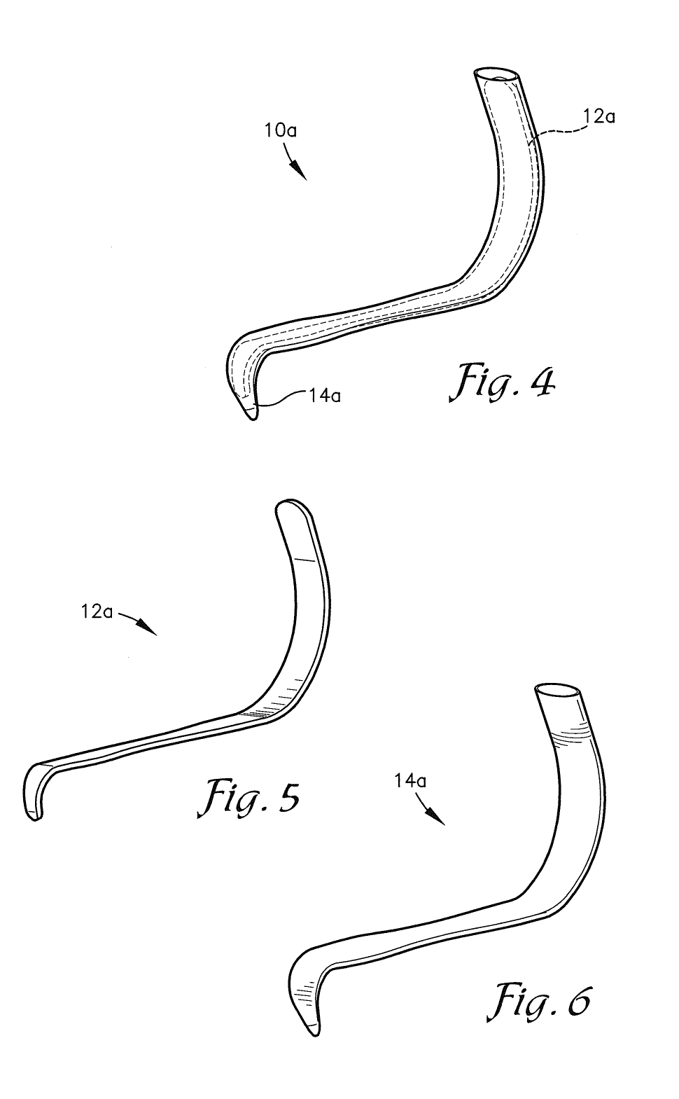

FIGS. 4-6 illustrate another embodiment of a surgical instrument 10a designed for use as a Taylor retractor, as used to hold back skin and tissue during various types of surgeries. The surgical instrument 10a may also comprise an internal stiffener 12a and an overmold component 14a. The surgical instrument 10a may be fabricated using the method steps described above in reference to surgical instrument 10, illustrated in FIGS. 1-3, but with different shapes and contours, as described below. Furthermore, the internal stiffener 12a and the overmold component 14a of FIGS. 4-6 may comprise the same materials and characteristics as described above in reference to surgical instrument 10, illustrated in FIGS. 1-3. The surgical instrument 10a may also comprise an x-ray marker (not shown), as described above.

As illustrated in FIGS. 4-6, the surgical instrument 10a may comprise a substantially lateral portion with a curved handle portion at one end thereof and a tapered and/or substantially pointy engagement portion at another end thereof. The handle portion may be curved in a direction opposite of a direction in which the engagement portion extends relative to the lateral portion. The internal stiffener 12a may have a substantially uniform thickness with contours substantially matching the contours of the overmold component 14a. The overmold component 14a may have varying thicknesses and rounded edges, particularly at locations thereof intended to contact tissue and/or bones of the patient during surgery, such as the engagement portion.

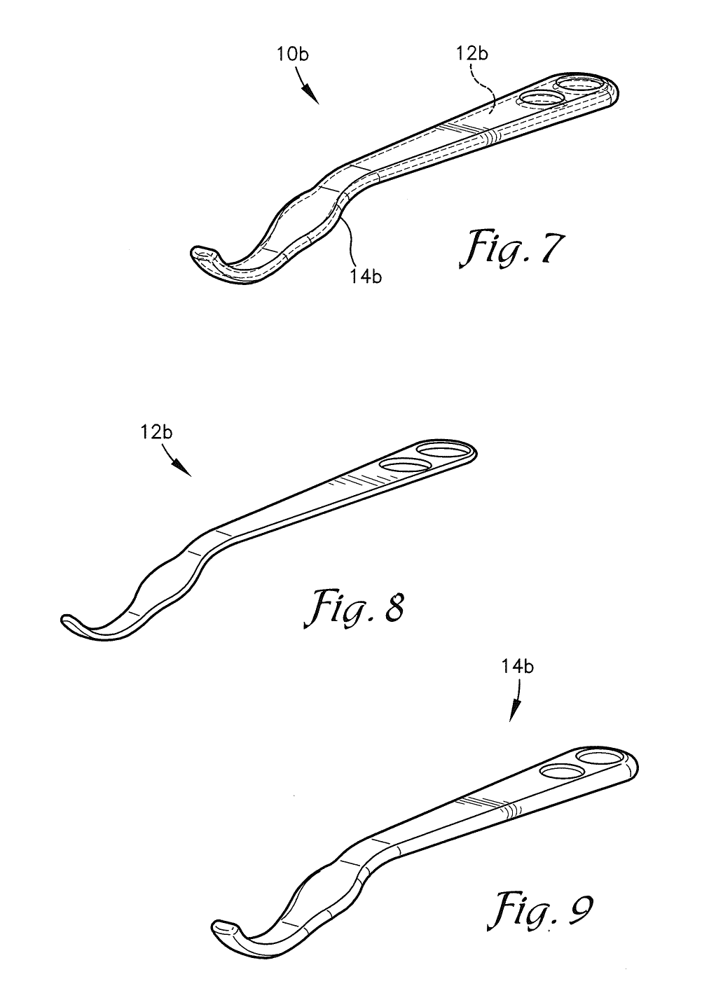

FIGS. 7-9 illustrate another embodiment of a surgical instrument 10b designed for use as a Hohmann retractor or a Hohmann blunt retractor, as used to retract tissue around bone during various surgeries. The surgical instrument 10b may also comprise an internal stiffener 12b and an overmold component 14b. The surgical instrument 10b may be fabricated as described above in reference to surgical instrument 10, illustrated in FIGS. 1-3, but with different shapes and contours, as described below. Furthermore, the internal stiffener 12b and the overmold component 14b of FIGS. 7-9 may comprise the same materials and characteristics as described above in reference to surgical instrument 10, illustrated in FIGS. 1-3. The surgical instrument 10b may also comprise an x-ray marker (not shown), as described above.

In some embodiments of the invention, the surgical instrument 10b may be fabricated to have a non-uniform distribution of thickness and flexibility. For example, the overmold component 14b, as illustrated in FIG. 7, may have a handle portion that is substantially tapered. The handle portion may additionally be formed in a tapering conical configuration (not shown). This may provide for a better grip than the traditional uniform thickness of prior art Hohmann retractors and may be gentler on tissue as it is removed and re [symbol] inserted into the patient. As illustrated in FIGS. 7-9, the surgical instrument 10b may also comprise a hook-like or curved engagement portion integral with the handle portion. The engagement portion may also taper toward an end thereof. For example, the handle portion may be tapered from a wide end to a narrow cross-section where it meets with the engagement portion. The engagement portion may then gradually widen in cross-section and then taper again to a smaller cross-section at the end opposite of the wide end of the handle. One or more holes may also be formed through the wide end of the handle portion of the surgical instrument 10b, as illustrated in FIGS. 7-9.

Although the invention has been described with reference to the embodiments illustrated in the attached drawing figures, it is noted that equivalents may be employed and substitutions made herein without departing from the scope of the invention as recited in the claims. For example, the methods of manufacturing surgical instruments, as described above in reference to Hibbs, Taylor, and Hohmann retractors, may be used to fabricate any type of retractors, forceps, elevators, dissectors, suction tubes, drill guides, separators, or any other retracting instruments used in the surgery of humans or animals. Specifically, a variety of instruments are known in the art for retracting, grasping, holding, occluding, dilating, probing, cannulating, or draining during surgery, and such instruments may also be fabricated using the methods and materials described above. Furthermore, the surgical instruments described herein may alternatively be made of one type of composite material formed as a single unitary instrument.

Having thus described various embodiments of the invention, what is claimed as new and desired to be protected by Letters Patent includes the following:

1. A radiolucent surgical instrument comprising:
an internal stiffener composed of a first composite material having a first modulus; and
an overmold component formed around at least a portion of the internal stiffener and composed of a second composite material having a second modulus, wherein the first modulus is greater than the second modulus.

2. The surgical instrument of claim 1, wherein the overmold component is formed around the internal stiffener via injection molding.

3. The surgical instrument of claim 1, wherein the internal stiffener is a structural laminate fabricated by composite ply lay-up and cure.

4. The surgical instrument of claim 1, wherein the bending stiffness of the internal stiffener is between approximately 10 MSI and 16 MSI.

5. The surgical instrument of claim 1, wherein the second composite material is a short fiber reinforced thermoplastic form.

6. The surgical instrument of claim 1, wherein the internal stiffener is connected to the overmold component via welding.

7. The surgical instrument of claim 1, wherein the overmold component has a non-uniform distribution of thickness.

8. The surgical instrument of claim 1, wherein the surgical instrument is at least one of a retractor, forceps, an elevator, a dissector, a suction tube, a drill guide, and a separator.

9. The surgical instrument of claim 1, further comprising an x-ray marker made of a non-radiolucent material, such that x-ray marker is locatable via x-ray.

10. A radiolucent surgical instrument comprising:
an internal stiffener composed of a first composite material having a first modulus and formed by way of composite ply lay-up; and
an overmold component formed around the internal stiffener by way of injection molding and composed of a second composite material having a second modulus, wherein the first modulus is greater than the second modulus.

11. The radiolucent surgical instrument of claim 10, wherein the internal stiffener is a structural laminate cured under heat and consolidation pressure.

12. The radiolucent surgical instrument of claim 10, wherein the bending stiffness of the internal stiffener is between approximately 10 MSI and 16 MSI.

13. The radiolucent surgical instrument of claim 10, wherein the second composite material is a short fiber reinforced thermoplastic form.

14. The radiolucent surgical instrument of claim 10, wherein the overmold component has a non-uniform distribution of thickness.

15. The surgical instrument of claim 10, wherein the surgical instrument is at least one of a retractor, forceps, an elevator, a dissector, a suction tube, a drill guide, and a separator.

16. The radiolucent surgical instrument of claim 10, wherein the surgical instrument is a Hohman retractor having a handle portion with a tapering cross-section.

17. The surgical instrument of claim 10, further comprising an x-ray marker made of a non-radiolucent material, such that x-ray marker is locatable via x-ray.

18. A method of forming a radiolucent surgical instrument, the method comprising:
forming an internal stiffener via composite ply lay-up and cure; placing the cured internal stiffener into a mold;
injecting composite material in liquid form into the mold and around the internal stiffener;
hardening the injected composite material into an overmold component of the surgical instrument; and
removing the mold from around the overmold component.

19. The method of claim 18, wherein a modulus of the internal stiffener is greater than a modulus of the overmold component.

20. The method of claim 18, further comprising bonding the internal stiffener with the overmold component via welding.

21. A radiolucent surgical instrument comprising:
an internal stiffener; and
an overmold component formed around at least a portion of the internal stiffener.
